(19) United States
(12) Patent Application Publication
(10) Pub. No.: US 2004/0072794 A1
(43) Pub. Date: Apr. 15, 2004

(54) Nutritional formulations containing synbiotic substances

(75) Inventors: Susan M. Kaup, Collingswood, NJ (US); Jeffrey L. Wilson, Doylestown, PA (US); Beverley M. Kostek, Glen Mills, PA (US); David C. Frantz, Pottstown, PA (US)

Correspondence Address: WYETH, PATENT LAW GROUP, FIVE GIRALDA FARMS, MADISON, NJ, 07940, US

(73) Assignee: Wyeth, Madison, NJ (US)

(21) Appl. No.: 10/681,658

(22) Filed: Oct. 8, 2003

Related U.S. Application Data
(60) Provisional application No. 60/418,109, filed on Oct. 11, 2002.

(51) Int. Cl. A61K 45/00; A61K 31/715

(52) U.S. Cl. 514/54; 424/93.45

(57) ABSTRACT

Nutritional compositions are provided which comprise oligofructose, sialyllactose and probiotic bacteria, which are useful in the eradication of pathogenic microorganisms in the gastrointestinal tracts of patients.

This application claims priority from co-pending provisional application No. 60/418,109, filed on Oct. 11, 2002, the entire disclosure of which is hereby incorporated by reference.

### FIELD OF THE INVENTION

This invention relates to nutritional formulations containing synbiotics and the use of such formulations in growth promotion of beneficial microorganisms and the inhibition and eradication of pathogenic organisms in the gastrointestinal tract of patients. More specifically, this invention relates to nutritional formulations containing oligofructose and sialyllactose in combination with specific strains of probiotic bacteria.

### BACKGROUND OF THE INVENTION

Synbiotics are “mixtures of probiotics and prebiotics that beneficially affect the host by improving the survival and implantation of live microbial dietary supplements in the gastrointestinal tract of the host.” (Andersson et al. “Health effects of probiotics and prebiotics: A literature review on human studies.” Scand. J. Nutr. 45:58-75 (2001)).

Prebiotics are nondigestible food ingredients that that beneficially affect the host by selectively stimulating the growth and/or activity of one or a limited number of bacterial species already established in the colon, and thus in effect improve host health. (Gibson et al., “Dietary modulation of the human colonic microbiota: Introducing the concept of prebiotics. J. Nutrition 125:167-176 (1995)).

Oligofructose is a well-known prebiotic. Oligofructose passes through the small intestine without being digested, reaching the large intestine. In the large intestine, oligofructose is fermented only by a limited range of microorganisms that include most species of Bifidobacteria, i.e., species of bacteria beneficial for human health. (See, e.g., Bouhnik et al, “Short Chain Fructo-Oligosaccharide Administration Dose-Dependently Increases Fecal Bifidobacteria in Healthy Humans,” J. Nutrition, 129:113-116 (1999)). Oligofructose is known to be a specific substrate for Bifidobacteria. (See, e.g., Mitsuoka et al, “Effect of Fructo-oligosaccharides on Intestinal Microflora”, Die Nahrung, 3, 5-6: 427-436 (1987)). Bifidobacteria produce short chain fatty acids as by-products of their metabolism, resulting in a reduction of the pH of the digestive tract.

U.S. Pat. No. 5,849,324 discloses a method for reducing the incidence of otitis media by enterally administering an effective amount of an indigestible nitrogen-free oligosaccharide. Specific oligosaccharides cited are those of fructose, xylose and galactose.

U.S. Pat. No. 5,827,526 discloses a method for reducing the duration of diarrhea by enterally administering on a prophylactic basis an effective amount of an indigestible nitrogen-free oligosaccharide.

U.S. Pat. No. 5,688,777 discloses a method for inhibiting infection by Clostridium difficile by enterally administering an effective amount of an indigestible nitrogen-free oligosaccharide. Administration of specific fructose oligosaccharides reduced or eliminated C. difficile, measured by stool colony forming units (cfu), in infected mice.

Sialyllactoses are oligosaccharides comprising a sialic acid and the disaccharide lactose. Sialic acids are a family of amino sugars containing 9 or more carbon atoms that are N- and O-substituted derivatives of neuraminic acid. The most common species of sialic acid is N-acetyineuraminic acid.

Sialyllactoses occur naturally in human milk as well as in milk of other mammals. However, sialyllactoses are present at noticeably higher concentrations in human milk compared to other mammalian species. The two primary species of sialyllactose in milk are 3′-sialyllactose and 6′-sialyllactose. These species occur naturally in human milk at a relative ratio of 1:3 (3′:6′).

Sialyllactose is known to have anti-adhesive properties for specific pathogenic bacteria. For example, sialyllactose acts to inhibit cholera toxin invitro (Idota et al., “Inhibition of Cholera Toxin by Human Milk Fractions and Sialyllactose,” Biosci. Biotech. Biochem. 59:417-419) and Helicobacter pylori (Simon et al., “Inhibition of Helicobacter pylori Binding to Gastrointestinal Epithelial Cells by Sialic Acid-Containing Oligosaccharides,” Infection and Immunity, 750-757, (1997)).

In light of its anti-adhesive properties, sialyllactose has been used to treat a number of medical conditions. For example, U.S. Pat. No. 5,260,280 discloses a composition containing sialic acid-containing oligosaccharides that neutralizes the effects of bacterial enterotoxin. U.S. Pat. Nos. 5,514,660, 5,753,630 and 5,883,079 disclose methods for treating or preventing an ulcer in the stomach or duodenum or inhibiting Helicobacter pylori infection, respectively, by administering an effective amount of a sialic acid-containing oligosaccharide. U.S. Pat. No. 5,620,965 relates to compositions for inhibiting binding of the bacterium Helicobacter pylori to stomach or duodenal cells by administering an effective amount of certain oligosaccharides.

U.S. Pat. No. 5,834,423 describes sialic acid derivatives that promote the proliferation of bifidobacteria and the use of effective amounts of certain sialylated oligosaccharides as an antidiarrheal agent. The sialylated oligosaccharides comprise 3′-sialyllactose and 6′-sialyllactose.

WO2001060346 discloses a nutritional composition comprising the prebiotic substances oligofructose and sialyllactose that act synergistically to stimulate the growth of the beneficial bifidobacteria.

Probiotics are live microbial food ingredients that have a beneficial effect on human health. (Salminen et al., “Functional food science and gastrointestinal physiology and function.” Brit. J. Nutr. 80(suppl. 1):S147-S171 (1998)).

Probiotic bacteria most commonly are “lactic acid bacteria”, so-called because they ferment carbohydrate to lactic acid. The specific strains most often studied include members of the genera Lactobacillus and Bifidobacterium. (Sanders, “Probiotics.” Food Technol. 53:67-77 (1999)).

Some lactic acid bacteria specifically produce lactic acid as a major product of their metabolism. Some produce predominantly the levorotary “L”-form of lactic acid [L(+)-lactic acid], others produce predominantly the dextrorotary “D”-form of lactic acid, while others produce both D-lactic acid and L-lactic acid. L(+)-lactate is a normal intermediary of mammalian metabolism. L(+)-lactate is oxidized rapidly and efficiently by the liver, kidney and brain. In contrast, D(−)-lactate is not well utilized by mammalian tissues and may lead to acidosis in the human infant.

Lactobacillus casei species strain GG, a probiotic bacterium commonly referred to as “Lactobacillus GG” or “LGG”, produces predominantly the levorotary L-form of lactic acid [L(+)-lactic acid]. LGG is found in the feces of infants and young children following oral administration. (Sepp et al., “Effect of administration of Lactobacillus casein strain GG on the gastrointestinal microbiota of newborns.” Microb. Ecol. Health Dis. 6:309-314 (1997); Sheen et al., “Short Report: A placebo-controlled study of Lactobacillus GG colonization in one-to-three-year-old Peruvian children.” Am. J. Trop. Med. Hyg. 52:389-392 (1995)).

A milk product containing LGG significantly shortened the duration of diarrhea in young children. (Kaila et al., “Enhancement of the circulating antibody secreting cell response in human diarrhea by a human Lactobacillus strain.” Pediatr. Res. 32:141-144 (1992); Isolauri et al., “The human Lactobacillus strain (Lactobacillus casei sp strain GG) promotes recovery from acute diarrhea in children. Pediatrics. 88:90-97 (1991)).

Lactobacillus acidophilus produces approximately equal amounts of D(−)-lactate and L(+)-lactate. Fermented milk containing L. acidophilus (strain CRL730) and L. casei (strain CRL431) eliminated diarrhea disease in four days on average in infants with post-gastroenteritis syndrome. The fermented milk restored the fecal flora to a predominantly lactic acid flora. (Gonzalez et al., “Biotherapeutic role of fermented milk.” Biotherapy. 8:129-134 (1995)).

U.S. Pat. No. 5,908,646 discloses a method for inhibiting the growth or activity of Clostridium species in a human food product by adding an effective amount of the beneficial microorganism, L. rhamnosus [L. casei subspecies rhamnosus], which produces predominantly L(+)-lactic acid.

U.S. Pat. No. 5,902,578 relates to a composition containing viable cells of three specific microorganisms beneficial to the human intestinal microorganisms for preventing diarrhea. Specifically, the three microorganisms are Lactobacillus reuteri, Lactobacillus acidophilus and Bifidobacterium infantis. The diarrhea can be caused by antibiotic treatment or by infection with a virus, a bacterium (e.g., E. coli) or a parasite.

U.S. Pat. No. 5,716,615 relates to a composition containing several different bacteria for treating gastrointestinal disorders. The microorganisms can be selected from lyophilized lactobacillus species, including L. acidophilus, lyophilized bifidobacterium species, including B. longum, B. infantis and B. bifidum, and Streptococcus thermophilus.

B. lactis is an L(+)-lactic acid producing bacteria. Historically, B. lactis Bb-12 was identified in the literature as “Bifidobacterium bifidum.” (Fukushima et al., “Effect of a probiotic formula on intestinal immunoglobulin A production in healthy children.” Int. J. Food Microb. 42-39-44 (1998)).

An unfermented infant formula containing both B. bifidum and Streptococcus thermophilus reduced the incidence of acute diarrhea and rotavirus shedding. (Saavedra et al., “Feeding of Bifidobacterium bifidum and Streptococcus thermophilus to infants in hospital for prevention of diarrhoea and shedding of rotavirus.” The Lancet. 344:1046-49 (1994)).

A fermented infant formula containing both B. bifidum and S. thermophilus induced a higher prevalence of bifidobacteria colonization of the bowel and a lower stool pH than an unfermented control formula. (Langhendries et al., “Effect of a fermented infant formula containing viable Bifidobacteria on the fecal flora composition and pH of healthy full-term infants.” J. Pediatr. Gastroenterol. Nutr. 21:177-181 (1995)).

Unfermented formulas containing both B. bifidum and S. thermophilus supported catch-up growth in malnourished children. Milk-based formulas containing the two bacteria induced probiotic colonization of the bowel. (Haschke et al (1998)) “Clinical trials prove the safety and efficacy of the probiotic strain Bifidobacterium Bb12 in follow-up formula and growing-up milks.” Monatsschr. Kinderheilkd. 146:S26-30 (1998).

WO2000010582 discloses compositions and methodologies for the utilization of one or more species or strains of lactic acid-producing bacteria, preferably strains of Bacillus coagulans, for the control of gastrointestinal tract pathogens, including antibiotic-resistant gastrointestinal tract pathogens, and their associated diseases.

Feeding an infant formula containing the probiotic B. bifidum (actually B. lactis Bb12) and prebiotic galacto-oligosaccharides to normal infants yielded a more favorable stool flora, with less Clostridia and more bifidobacteria. (Fukushima et al. “Effect of follow-up formula containing Bifidobacteria (NAN BF) on fecal flora and fecal metabolites in healthy children.” Bloscience Microflora. 16:65-72 (1997)).

U.S. Pat. No. 6,241,983 discloses a composition containing beneficial human intestinal microorganisms and a source of dietary fiber for promoting gastrointestinal health. More specifically, the microorganism can be selected from lactobacillus and bifidobacterium species. The sources of dietary fiber include pentosans, beta.-glucans, pectins and pectic polysaccharides, mannans, arabinans and galactans, fructose oligosaccharides, and mixtures thereof.

U.S. Pat. No. 5,744,134 claims a composition containing beneficial human intestinal microorganism and a source of dietary fiber for promoting gastrointestinal health. More specifically, the microorganism can be selected from lactobacillus and bifidobacterium species. The sources of dietary fiber are inulin, fructose oligosaccharides, pectin, guar gum and mixtures thereof.

WO2001015714 discloses a compostion useful for enhancing general immunity. The composition includes one or more micronutrients, one or more compounds selected from the group of a prebiotic, probiotic, and synbiotic, and lipid-based or carbohydrate-based excipient.

WO2000033854 describes a preparation having a health-promoting action, in particular for the prevention and/or treatment of disorders of the digestive tract, which contains one or more probiotics and one or more non-digestible oligosaccharides. The probiotics are preferably chosen from bacterial strains such as a strain of a Lactobacillus or a Bifidobacterium species and from yeast strains such as a strain of a Saccharomyces species. The prebiotics can include hydrolyzed carob gum, inulin, arabinogalactan, arabinoxylan, beta-glucan, L-arabinan, galactomannan and glucomannan.

EP 904784 discloses a nutritional preparation with health-promoting action, in particular with respect to the prevention and treatment of disorders of the gastrointestinal tract, comprising an effective amount of viable cells of each of the following microorganisms: Bifidobacterium; Enterococcus faecium; and a Lactobacillus strain that produces predominantly levorotary L(+)-lactate. Exemplary Bifidobacterium species include B. infantis and B. lactis.

### SUMMARY OF THE INVENTION

The present invention is related to nutritional compositions comprising oligofructose, sialyllactose and probiotic bacteria. The present invention is further directed to a method of inhibiting or eradicating pathogenic organisms in the gastrointestinal tract of patients, comprising enterally administering to said patient a composition comprising oligofructose, sialyllactose and probiotic bacteria.

### DETAILED DESCRIPTION OF THE INVENTION

The present inventors have found that the combination of oligofructose, sialyllactose and probiotic bacteria eradicates intestinal infection with pathogenic bacteria, particularly enteropathogenic E. coli, and may therefore be used for the prophylaxis of diarrhea due to enteropathogenic E. coli. Preferred probiotic bacteria for use in the present formulations include L. acidophilus and B. lactis.

The sialyllactose useful in the present compositions comprises 3′-sialyllactose and 6′-sialyllactose. Preferably, the sialyllactose used herein is 3′-sialyllactose.

The sialyllactose may be prepared according to any of the methods described, e.g., in U.S. Pat. Nos. 5,575,916; 5,714,075; 5,278,299; 5,374,541; and 5,876,980. However, it will be recognized by those skilled in the art that any other method of synthesizing and purifying sialyllactose may be useful to prepare the sialyllactose used in the present compositions.

The oligofructose useful in this invention may be prepared by any known method of synthesis and/or isolation. A commercially available form of oligofructose useful in this invention is Raftilose® available from Orafti S. A., Tienen, Belgium.

Oligofructose comprises a series of oligosaccharides found naturally in vegetables, such as onion and the root of the chicory plant. Oligofructose may be prepared industrially from a naturally occurring polyfructose (inulin) which may be found in many plants, including onions, leeks, wheat, chicory and Jerusalem artichoke. Chicory is most commonly used. Oligofructose can be recovered in sufficient quantities from these plants by methods known in the art. The naturally occurring inulin comprises oligofructose and higher polymers of fructose. Oligofructose derived from inulin from plants such as chicory contains both polyfructose chains and polyfructose chains with a terminal glucose unit.

Oligofructose may be prepared by synthesis rather than by extraction procedures. Oligofructose may be synthesized from sucrose by transfructosylation, which is accomplished by means of an enzyme, β-fructofuranosidase, which links additional fructose monomers to the sucrose molecule. Oligofructose formed in this manner contains fructose units linked to a terminal glucose unit. Examples of such fructose oligosaccharides are kestose (GF₂), nystose (GF₃) and fructofuranosyl nystose (GF₄). An oligofructose comprising a mixture of oligosaccharides prepared by methods such as these is NutraFlora®, available from GTC Nutrition Company, Golden, Colo, USA.

Suitable probiotics useful in the present invention are Bifidobacterium and Lactobacillus. Bifidobacterium lactis BB1 and Lactobacillus acidophilus NCFM® are available from Rhodia Inc.

The nutritional compositions of the present invention may comprise (or may be capable of comprising after dilution with water) 0.1 g/L to 10 g/L of oligofructose; 6 mg/L to 10 g/L of sialyllactose; 10⁶ to 10¹⁴ colony forming units (cfu) per liter of Lactobacillus; and 10⁶ to 10¹⁴ cfu/L of Bifidobacterium. Preferably, the present compositions comprise (or are capable of comprising after dilution with water) 0.3 g/L to 6 g/L of oligofructose; 60 mg/L to 1 g/L of sialyllactose; 10⁸ to 10¹² cfu/L of Lactobacillus; and 10⁸ to 10¹² cfu/L of Bifidobacterium. More preferably the present formulations comprise (or are capable of comprising after dilution with water) 1 g/L to 3 g/L of oligofructose; 100 mg/L to 600 mg/L of sialyllactose; 10⁹ to 10¹¹ cfu/L of Lactobacillus; and 10⁹ to 10¹¹ cfu/L of Bifidobacterium and even more preferably about 3 g/L of oligofructose; about 100 mg/L of sialyllactose; about 3×10¹⁰ cfu/L of Lactobacillus; and about 3×10¹⁰ cfu/L of Bifidobacterium.

The nutritional compositions of the present invention can be utilized in combination with or in the form of various nutritional products, such as infant formula, follow-on formula, toddler's beverage, milk, yogurt, fruit-based products for older children (such as fruit juices) candies, chewing gum, lozenges, powders, tablets, etc. Preferably, the present nutritional compositions are used in the form of an infant formula. When used as an infant formula, it may be in the form of a ready to feed liquid or a powder, which may be mixed with water and fed to the infant. It is most preferred that the present formulation be added to infant formula in powder form.

Infant formula suitable for use with the present invention should contain all vitamins and minerals considered essential in an infant's daily diet. These vitamins and minerals should be present in nutritionally significant amounts. Examples of vitamins, minerals and other nutrients which may be included in infant formulas in which the present formulations are to be added include vitamin A, vitamin B complex, vitamin C, vitamin D, vitamin E, vitamin K, calcium, magnesium sodium, potassium, phosphorus, copper, zinc, chloride, iodine, selenium, iron, niacin, folic acid, pantothenic acid, biotin, choline, inositol and manganese.

The infant formula may contain one or more lipid sources as will be recognized by those skilled in the art. The infant formula may further contain other substances known to have a beneficial effect. Examples of such substances include nucleotides, immunoglobulins, polyunsaturated fatty acids, etc.

The present invention is further illustrated with reference to the following non-limiting example.

### EXAMPLE 1

A preferred infant formula according to the present invention provides the following nutrients when 127.3 grams of said infant formula are diluted to a volume of one liter with water:

| Nutrient | Units | per Liter |
|---|---|---|
| Energy | Kcal | 672 |
| Protein | g | 15 |
| Whey: Casein ratio | 60-40 | |
| Fat | g | 36 |
| Carbohydrate, including | g | 72 |
| Oligofructose | g | 3.0 |
| Sialyllactose | mg | 100 |
| L. acidophilus NCFM | cfu | 3 × 10¹⁰ |
| B. lactis BB1 | cfu | 3 × 10¹⁰ |
| Vitamin A | RE | 750 |
| Mixed natural Carotenoids | IU | 400 |
| Vitamin D | mcg | 10.6 |
| Vitamin E | IU | 7.4 |
| Vitamin K | mcg | 67.0 |
| Vitamin B1 (thiamin) | mcg | 1000 |
| Vitamin B2 (riboflavin) | mcg | 1500 |
| Vitamin B6 (pyridoxine) | mcg | 600 |
| Vitamin B12 (cyanocobalamin) | mcg | 2.0 |
| Niacin | mcg | 9.0 |
| Folic Acid | mcg | 80 |
| Pantothenic Acid | mcg | 3000 |
| Biotin | mcg | 90 |
| Vitamin C (ascorbic acid) | mg | 20 |
| Choline | mg | 100 |
| Inositol | mg | 33 |
| Calcium | mg | 460 |
| Phosphorus | mg | 333 |
| Magnesium | mg | 64 |
| Iron | mg | 8.0 |
| Zinc | mg | 6.0 |
| Manganese | mcg | 50 |
| Copper | mcg | 560 |
| Iodine | mcg | 100 |
| Sodium | mg | 160 |
| Potassium | mg | 650 |
| Chloride | mg | 433 |
| Selenium | mcg | 14 |

The following experiment illustrates the effectiveness of Lactobacillus acidophilus and Bifidobacterium lactis. to eradicate intestinal infection with enteropathogenic E. coli.

### EXPERIMENT

The following experiment was designed to evaluate a combination of Lactobacillus acidophilus and Bifidobacterium lactis. as a means of prophylaxis of diarrhea due to enteropathogenic E. coli (“EPEC”) by intentionally infecting infant monkeys with this pathogen.

Newborn infant rhesus monkeys (Macaca mulatto) were fed either, on an exclusive basis, humanized infant formula (S26, available from Wyeth Nutrition) or breast milk from birth.

At the begining of eight (8) to nine (9) weeks of age, the monkeys were given 10⁹ colony forming units (cfu) of enteropathic Escherichia coli (EPEC) E2348/69. The EPEC was administered either in the humanized infant formula or the breast milk by orogastric intubation. Stool consistency, appetite, body temperature and dehydration were assessed. Rectal swabs were obtained from each monkey on the day of EPEC administration and at 3,6, and 19 or 21 days past inoculation.

A probiotic was prepared by blending 150 grams of an equal blend of L. acidophilus NCFM® (a trademark of the North Carolina Dairy Foundation) and Bifidobacterium infantis BB1 (both obtained from Rhodia Inc.) containing 10¹⁰ cfu/g of each microorganism. The probiotic was incorporated into the humanized formula only. Seven days prior to the conclusion of the study, all of the formula-fed monkeys were switched to formulas supplemented with the probiotic mixture described above. The probiotic was fed at a titer of 1.3×10¹⁰ cfu/L of each bacterium.

Rectal swabs were collected and a microbial assessment performed as described below.

In order to assess colonization of the exogenously introduced pathogenic and probiotic bacteria, a polymerase chain reaction (PCR) assay was developed which was capable of identifying specific microbial species. The primers were designed to detect only the species of interest to the exclusion of other species in the genus. DNA was isolated from two sets of samples of fecal cultures grown on LAC25 plates, one set grown aerobically and a second set grown anaerobically. Both sets of fecal cultures were subsequently frozen. PCR reactions were carried out as follows: 94° C. for 30 seconds, 50° C. for 1 minute, and 72° C. for 1 minute for 5 cycles immediately followed by 94° C. for 30 seconds, 56° C. for 1 minute, and 72° C. for 1 minute for 30 cycles. A final extension period (10 minutes at 72° C.) was incorporated to ensure complete synthesis of all DNA products. PCR products were separated by agarose gel electrophoresis and visualized by transillumination.

Identification of both B. lactis and L. acidophilus was confirmed by 16S ribosomal techniques. Regions of the 16S rRNA gene of specific length from B. lactis and L. acidophilus were PCR amplified from genomic DNA isolated from bacterial colonies. Because of the homology of the 16S rRNA gene across species, primers were designed from E. coli (positions 005 and 531) which specifically generated identifiable products of unique length from the species of interest. Cycle sequencing of the 16S rRNA amplification products was carried out using AmpliTaq FS DNA polymerase and dRhodamine dye terminators and were electrophoresed on a ABI Prism 377 DNA sequencer. Data were analyzed using PE/Applied Biosystems DNA editing and assembly software. The sequences from the amplification products were compared to the sequence database and yielded a 16S rRNA sequence homology of greater than 99% accuracy which is indicative of a species level match.

Primer pairs were designed based on these identifications. Public DNA databases were consulted to determine DNA regions of the bacteria which were appropriate for unique PCR primer pairs. The B. lactis primer pairs were targeted to a 413 bp region of B. lactis while the L. acidophilus primer pairs were targeted to a 460 bp region of L. acidophilus. In order to facilitate identification on agarose gels, the primers for L. acidophilus were modified by the addition of GC-tails.

Primer pairs designed to specifically detect L. acidophilus were validated against L. rhamnosus, L. plantarum, and L. rhamnosus GG. Primer pairs designed to specifically detect B. lactis were validated against B. adolescentis, B. infantis, and B. bifidum. Only the species of interest was detected in each instance.

PCR methodology was also developed and used to determine whether the animals had been colonized by exogenously introduced enteropathogenic E. coli (EPEC) E2348/69. The PCR method consisted of isolating DNA from each of the sixty frozen samples prior to the PCR reaction described above. Based on published sequence information, the EPEC PCR primers were designed to specifically detect a 326 bp region of the gene encoding the BFP protein of EPEC. EPEC Primers designed to detect EPEC were validated against enterotoxigenic E. coli (ETEC). Only EPEC was detected.

Fecal swab samples from the monkeys were examined for the presence of EPEC DNA by PCR. EPEC was not detected in any of the samples taken on treatment day 0, the day of infection. On treatment day 3 (post-infection with EPEC), all samples tested positive for EPEC. Surprisingly, on the final day of the study, treatment day 19 or 21, no EPEC was detected from any of the fecal swab samples taken from the probiotic supplemented formula-fed monkeys; whereas EPEC was detected in all the fecal swab samples from the breast-fed monkeys.

Table 1 sets forth the results of the PCR determination. Monkeys innoculated with EPEC had EPEC-positive swabs for up to three weeks after the single EPEC innoculation. This suggests successful colonization of these monkeys. The monkeys fed the formula supplemented with probiotics after EPEC-innoculation and colonization had EPEC-negative fecal swabs by the end of the study. In contrast, breast-fed monkeys which received no probiotics remained EPEC-positive.

TABLE 1

| | | | | |
|---|---|---|---|---|
| Animal ID | Day | EPEC | B. lactis | L. acidophilus |

Formula-fed Monkeys

| | | | | |
|---|---|---|---|---|
| 47 | 0 | − | ND | ND |
| | 3 | + | ND | ND |
| | 6 | + | − | − |
| | 19 | − | + | + |
| 48 | 0 | − | ND | ND |
| | 3 | − | ND | ND |
| | 6 | + | − | − |
| | 19 | − | + | − |
| 40 | 0 | − | ND | ND |
| | 3 | + | ND | ND |
| | 6 | + | − | − |
| | 19 | − | + | − |
| 57 | 0 | − | ND | ND |
| | 3 | + | ND | ND |
| | 6 | + | − | − |
| | 19 | − | + | + |
| 63 | 0 | −/+ | ND | ND |
| | 3 | + | ND | ND |
| | 6 | + | − | − |
| | 19 | − | + | + |
| 91 | 0 | − | ND | ND |
| | 3 | + | ND | ND |
| | 6 | + | − | − |
| | 19 | − | + | + |
| 100 | 0 | − | ND | ND |
| | 3 | + | ND | ND |
| | 6 | + | + | − |
| | 21 | − | + | − |
| 105 | 0 | − | ND | ND |
| | 3 | + | ND | ND |
| | 6 | + | + | − |
| | 21 | − | + | − |
| 109 | 0 | − | ND | ND |
| | 3 | + | ND | ND |
| | 6 | + | − | − |
| | 21 | − | + | − |

Breast-fed monkeys

| | | | | |
|---|---|---|---|---|
| 118 | 0 | − | ND | ND |
| | 3 | + | ND | ND |
| | 6 | + | + | − |
| | 21 | + | − | + |
| 108 | 0 | − | ND | ND |
| | 3 | + | ND | ND |
| | 6 | + | + | − |
| | 21 | −/+ | + | − |
| 54 | 0 | − | ND | ND |
| | 3 | + | ND | ND |
| | 6 | + | + | − |
| | 21 | + | + | − |

ND=not done;

−/+ indicates varying results among duplicates

The intestinal colonization of the probiotics (L. acidophilous NCFM and Bifidobacteria lactis BBI) was confirmed by PCR analysis as described above, after the probiotic supplementation regimen in the formula-fed monkeys was completed. Table 1 demonstrates that enternally administered human probiotics colonized the infant rhesus monkeys. Concurrently, no pathogenic E. coli was detected from isolates obtained from the gastrointestinal tracts of the monkeys.

The infant formula of Example 1 may be used similarly to eradicate pathogenic E. coli from the gastrointestinal tracts of monkeys.

The present invention may be embodied in other specific forms without departing from the spirit and essential attributes thereof and accordingly, reference should be made to the appended claims, rather than to the foregoing specification as indicating the scope of the invention.

What is claimed is:

1. A nutritional composition comprising oligofructose, sialyllactose and probiotic bacteria.

2. A nutritional composition as in claim 1 wherein the probiotic bacteria is Lactobacillus acidophilus and Bifidobacterium lactis.

3. A nutritional composition as in claim 2, comprising, or being capable of comprising after dilution with water, 0.1 g/L to 10 g/L of oligofructose; 6 mg/L to 10 g/L sialyllactose; 10⁶ to 10¹⁴ cfu/L of L. acidophilus and 10⁶ to 10¹⁴ cfu/L of B. lactis.

4. A method of eradicating pathogenic organisms in the gastrointestinal tract of a patient in need thereof, comprising enterally administering to said patient a nutritional composition comprising oligofructose, sialyllactose and probiotic bacteria.
